(19) United States
(12) Patent Application Publication
(10) Pub. No.: US 2010/0209530 A1
(43) Pub. Date: Aug. 19, 2010

(54) ANTI-ALLERGEN AGENT

(76) Inventor: Yoshinao Yamada, Aichi (JP)

Correspondence Address: BIRCH STEWART KOLASCH & BIRCH, PO BOX 747, FALLS CHURCH, VA, 22040-0747, US

(21) Appl. No.: 12/680,795

(22) PCT Filed: Sep. 24, 2008
(86) PCT No.: PCT/JP2008/067138

§ 371 Date: Mar. 30, 2010

(30) Foreign Application Priority Data
Oct. 1, 2007 (JP) 2007-258131
Oct. 18, 2007 (JP) 2007-271839

(51) Int. Cl. A61K 33/42; A61K 33/06; A61K 33/24; C01B 25/26; C01B 39/38; C01G 30/00

(52) U.S. Cl. 424/601; 424/604; 424/684; 424/617; 423/308; 423/700; 423/617

(57) ABSTRACT

The present invention provides an anti-allergen agent that has excellent heat resistance, low coloration, excellent processability, and excellent water resistance, an anti-allergen product, and a method for processing same. The anti-allergen agent of the present invention comprises as an active ingredient an inorganic solid acid, and the inorganic solid acid preferably has an acid strength as pKa of 4.0 or less. Furthermore, it preferably further comprises a polyphenol compound, and in this case it preferably comprises the inorganic solid acid at 5 to 90 wt % relative to the total amount of the inorganic solid acid and the polyphenol compound.

### TECHNICAL FIELD

The present invention relates to an anti-allergen agent and an anti-allergen product.

### BACKGROUND ART

In recent years, the number of people suffering from allergic diseases such as hay fever due to Cryptomeria japonica pollen, etc., bronchial asthma due to house dust caused by mites, etc., pollen allergy, allergic rhinitis, and atopic dermatitis has been increasing, which is a serious problem. As methods for treating such allergic diseases, there has been great progress as a result of the development of the series of medicinal agents called anti-allergy agents, and inhaled or external steroids, but these are still only symptomatic treatments and not curative treatments.

Furthermore, a miticide, etc. is generally used for the eradication of house dust mites, but Dermatophagoides farinae, Dermatophagoides pteronyssinus, etc. in house dust have the characteristics that not only the mite's bodies but also the feces and remains thereof cause an allergen reaction, and since fine allergen particles are gradually released as the remains decompose after death, merely killing mites cannot inactivate the allergens. Moreover, masks are used for preventing inhalation of the pollen of Cryptomeria japonica, etc., but since the allergen activity of pollen attached to the mask does not disappear, there is a risk of inhalation when the pollen is scattered again.

Because of such problems, in order to alleviate the symptoms of an allergic disease or prevent new sensitization, it is necessary to remove allergens, which are the substances causing allergic symptoms, from a living space before they are inhaled into the human body, or render them harmless by modification.

As a method for removing an allergen without using a medicinal agent, there is a method in which the allergen is lessened by physically removing floor-deposited dust or air-suspended dust by suction with a vacuum cleaner or an air purifier. However, a large amount of allergen sucked up by a vacuum cleaner is simply stored in a dust bag, and it can expected that there will be a risk of rescattering the allergen when disposing of the dust bag. Furthermore, it is difficult to completely remove a fine particulate substance by removal using an air purifier, and there is a risk of rescattering.

Consequently, an anti-allergen agent that inactivates a harmful allergen to make it harmless by the action of adsorption on or covering of a reactive site of the allergen with an antibody has recently been proposed. For example, methods employing tannic acid (ref. e.g. Patent Documents 1 and 2 and Non-Patent Document 1) or polyphenols such as tea extract and gallic acid, which are analogous compounds to tannic acid (ref. Patent Document 3) are known. However, an organic allergen reducing agent such as tannic acid is chemically unstable, and there are the problems that when it is attached to fiber or fiber products, coloration or discoloration over time might occur, and it might run out to the environment due to water, oil, solvent, or washing, thus staining clothing or causing skin irritation. Experiment 2 of Patent Document 1 discloses that tannic acid can be removed by distilled water, and it is thus clear that when fiber treated with tannic acid is washed repeatedly tannic acid is lost. Therefore, there is a problem with the use thereof as an anti-allergen agent in fiber or fiber products that have a possibility of becoming wet, being washed, or being in direct contact with the skin, and there is the drawback that application targets are limited for fiber products that are exposed to view because of problems with color tone, heat resistance, or durability. There is therefore a desire for the development of an anti-allergen agent that eliminates the above-mentioned defects.

(Patent Document 1) JP-A-61-44821 (JP-A denotes a Japanese unexamined patent application publication)
(Patent Document 2) JP-B-2-16731 (JP-B denotes a Japanese examined patent application publication)

### (Patent Document 3) JP-A-6-279273

(Non-Patent Document 1) ‘Sosetsu Tanninnikansuru Saikinnokenkyu’ (Review of Recent Research into Tannin), Yakugaku Zasshi, 103 (2), 125-142 (1983)

### DISCLOSURE OF INVENTION

### Problems to be Solved by the Invention

In light of the above-mentioned circumstances, the object of the present invention is to provide an anti-allergen agent that has excellent heat resistance, low coloration, excellent processability, and excellent water resistance, an anti-allergen product, and a method for processing same.

### Means for Solving the Problems

As a result of an intensive investigation by the present inventors in order to solve the above-mentioned problems, it has been found that the above-mentioned problems can be solved by means described in <1> and <7> to <9> below. They are described below together with <2> to <6>, which are preferred embodiments.

<1> An anti-allergen agent comprising as an active ingredient an inorganic solid acid,
<2> the anti-allergen agent according to <1> above, wherein the inorganic solid acid has an acid strength as pKa of 4.0 or less,
<3> the anti-allergen agent according to <1> or <2> above, wherein the inorganic solid acid is at least one selected from the group consisting of zirconium phosphate, aluminum phosphate, tin phosphate, cerium phosphate, titanium phosphate, an H-exchanged Y type zeolite, an H-exchanged ZSM-5 type zeolite, antimonic acid, an SiO₂—Al₂O₃ composite oxide, an SiO₂—TiO₂ composite oxide, an SiO₂—ZrO composite oxide, an SiO₂—Ga₂O₃ composite oxide, a TiO₂—Al₂O₃ composite oxide, a TiO₂—ZrO composite oxide, a TiO₂—SnO composite oxide, a TiO₂—ZnO composite oxide, and magnesium silicate,
<4> the anti-allergen agent according to any one of <1> to <3> above, wherein the anti-allergen agent further comprises a polyphenol compound,
<5> the anti-allergen agent according to <4> above, wherein the inorganic solid acid is contained at 5 to 90 wt % relative to the total amount of the inorganic solid acid and the polyphenol compound,
<6> the anti-allergen agent according to <4> or <5> above, wherein the polyphenol compound is tannic acid,
<7> an anti-allergen composition comprising the anti-allergen agent according to any one of <1> to <6> above,
<8> a method for processing an anti-allergen product, employing the anti-allergen composition according to <7> above, and
<9> an anti-allergen product that has been processed by the method for processing an anti-allergen product according to <8> above.

### BEST MODE FOR CARRYING OUT THE INVENTION

The present invention is explained below.

In the present invention, the allergen is not limited as long as an allergy is caused by humans or animals making skin contact or mucosal contact with the allergen, and specific examples thereof include allergens originating from the body hair or epithelium of dog, cat, bird, etc., allergens originating from a plant such as the pollen of Cryptomeria japonica, Japanese cypress, artemisia, zelkova, timothy grass, vernal grass, ragweed, etc. and natural rubber latex, allergens originating from molds, and animal or plant proteins such as mite and cockroach bodies or excrement. In general, house dust mite-derived allergens, which are often contacted as house dust inside houses, and pollen allergens, which are substances causing pollen allergy, are preferred.

The inorganic solid acid referred to in the present invention is a solid that is an inorganic substance and has on its surface a moiety (acid site or active site) that releases H⁺, thereby exhibiting acidity. Specific examples of the inorganic solid acid include zirconium phosphate, aluminum phosphate, tin phosphate, cerium phosphate, titanium phosphate, an H-exchanged Y type zeolite, an H-exchanged ZSM-5 type zeolite, antimonic acid, an SiO₂—Al₂O₃ composite oxide (commonly called silica-alumina), an SiO₂—TiO₂ composite oxide (commonly called silica-titania), an SiO₂—ZrO composite oxide, an SiO₂—Ga₂O₃ composite oxide, a TiO₂—Al₂O₃ composite oxide, a TiO₂—ZrO composite oxide, a TiO₂—SnO composite oxide, a TiO₂—ZnO composite oxide, magnesium silicate, and a special inorganic ion exchanger. Among them, zirconium phosphate, an H-exchanged ZSM-5 type zeolite, an H-exchanged Y type zeolite, and an SiO₂—Al₂O₃ composite oxide (commonly called silica-alumina) are preferred solid acids since they are formed from inorganic substances having excellent heat resistance and have high solid acidity. Among them, zirconium phosphate, which has high acid strength, is more preferable, and in particular lamellar zirconium phosphate, whose crystal system has a lamellar structure, is most preferable since its acid strength is particularly high.

With regard to the form of the inorganic solid acid in the present invention, there are powder form, clump form, tabular form, fiber form, etc., and in order for it to be applied to processing into various materials and configurations powder form is preferable. In the case of powder form, the average particle size is preferably 0.01 to 50 μm, and more preferably 0.02 to 20 μm. A powder having an average particle size of at least 0.01 μm has the advantage of ease of handling since it does not easily reaggregate and, furthermore, when dispersed in a surface treatment agent such as a binder and post-processed with a fiber, etc., particles having an average particle size of no greater than 50 μm are preferable since they have good dispersibility, do not degrade the texture of a fiber, and do not easily cause thread breakage when kneaded with a fiber.

There is no restriction on the color tone of the inorganic solid acid in the present invention, but in order to apply it to processing into various materials and configurations, white or a pale color having high lightness is preferable. The lightness is preferably at least 60% when black is defined as 0% and white as 100%.

The acid strength of the inorganic solid acid referred to in the present invention is the ability of an acid site of the inorganic solid acid surface to donate a proton to a base or the ability thereof to accept an electron pair from a base. Measurement of acid strength may be carried out by a method employing an acid-base indicator. Selecting an appropriate acid-base indicator as a base enables acid strength to be measured as the ability to convert the base form of the indicator into its conjugate acid form.

Examples of acid-base indicators that can be used in measurement of acid strength and their color-change pKa values include neutral red (+6.8), methyl red (+4.8), 4-phenylazo-1-naphthylamine (+4.0), dimethyl yellow (+3.3), 2-amino-5-azotoluene (+2.0), 4-phenylazo-diphenylamine (+1.5), 4-dimethylaminoazo-1-naphthalene (+1.2), crystal violet (+0.8), p-nitrobenzeneazo-p′-nitro-diphenylamine (+0.43), dicinnamylacetone (−3.0), benzalacetophenone (−5.6), and anthraquinone (−8.2). Use of such various acid-base indicators whose acid strength (pKa) is known enables acid strength to be measured. The lower the pKa value of the indicator whose color is changed, the higher the acid strength.

A method for measuring acid strength of an inorganic solid acid employing the above-mentioned acid-base indicators is as follows.

0.1 g of a solid acid is weighed in a test tube, and 2 mL of benzene is added thereto and mixed by gently shaking. Two drops of a 0.1% benzene solution of an indicator (0.1% ethanol solution for crystal violet) are added thereto and mixed by gently shaking, and change of color is examined.

The benzene solution containing an acid-base indicator exhibits an acid color on the acid side relative to the color-change pKa value of the acid-base indicator, exhibits a base color on the base side relative to the color-change pKa value of the acid-base indicator, and exhibits a mixed color of the acid color and the base color at the color-change pKa value and its vicinity (also called the ‘color-change range’) of the acid-base indicator.

When an acid-base indicator for which a color-change range is observed is found, the color-change pKa value of this acid-base indicator represents the acid strength of the inorganic solid acid. Furthermore, where there is no acid-base indicator for which a color-change range is observed, the acid strength (pKa value) of the inorganic solid acid is expressed as being lower than the acid strength of the acid-base indicator having the lowest acid strength among those for which an acid color is observed (color-change pKa value of acid-base indicator having the smallest color-change pKa value among those for which an acid color is observed) and being higher than the acid strength of the acid-base indicator having the highest acid strength among those for which a base color is observed (color-change pKa value of acid-base indicator having the largest color-change pKa value among those for which a base color is observed).

Furthermore, when there is no appropriate acid-base indicator that shows a lower limit, the pKa value of the inorganic solid acid is generally expressed as being smaller than (pKa value of acid-base indicator having the smallest color-change pKa value among those for which an acid color is observed), and when there is no appropriate indicator that shows an upper limit, the pKa value of the inorganic solid acid is generally expressed as being larger than (pKa value of acid-base indicator having the largest color-change pKa value among those for which a base color is observed).

With regard to the acid strength of the inorganic solid acid in the present invention, the pKa value is preferably low, since the lower it is, the higher the anti-allergen effect. Specifically, the pKa is preferably 4.0 or less, more preferably 3.3 or less, and yet more preferably 1.5 or less. Among them, a solid acid having a pKa of 1.5 or less has a particularly excellent anti-allergen effect, and exhibits a high effect toward various allergen substances. That is, the anti-allergen agent of the present invention is preferably an inorganic solid acid having a low pKa value.

Moreover, it is preferable for the pKa of the inorganic solid acid to be 4.0 or less since the anti-allergen effect when used in combination with a polyphenol compound is excellent.

The anti-allergen effect of the inorganic solid acid of the present invention is easily exhibited when it has a defined moisture content. An inorganic solid acid having hygroscopicity can retain moisture in the solid acid even when it is mixed with another material or the humidity of the atmosphere changes, and it is excellent in terms of having in the inorganic solid acid itself the moisture necessary to inactivate an allergen.

Furthermore, it is surmised that when used in combination with a polyphenol compound, the polyphenol compound is hydrated and swollen with moisture contained in the inorganic solid acid, and easily acts on a protein that is an allergen. In a conventional anti-allergen agent using a polyphenol compound on its own, the allergen-inactivating performance is weak in a moisture-free state, whereas if an excessive amount of moisture is added, since the polyphenol compound is washed away, there is a problem with water resistance. In the present invention, when an inorganic solid acid having a defined moisture content is used, since the polyphenol compound is retained together with the moisture, allergen inactivating performance is exhibited and the allergen inactivating performance is not degraded even when exposed to an excessive amount of water.

The anti-allergen agent of the present invention preferably comprises an inorganic solid acid and a polyphenol compound.

The polyphenol compounds in the present invention are organic compounds having a plurality of phenolic hydroxy groups (hydroxy groups bonded to an aromatic ring such as a benzene ring or a naphthalene ring) in the molecule. Among them, those that are industrially available at low cost are low molecular weight polyphenols, which are generally called catechins and comprise a mixture of epicatechin, gallotannin, epigallocatechin, epicatechin gallate, epigallocatechin gallate, etc., and high molecular weight tannic acid, and they are preferably used. In the present invention, tannic acid, which has high synergistic effect when used in combination with an inorganic solid acid, is more preferable.

The anti-allergen agent of the present invention employs an inorganic solid acid or employs in combination an inorganic solid acid and a polyphenol compound and, in the case of an inorganic solid acid on its own, is characterized by very high heat resistance and discoloration resistance. When an inorganic solid acid is used in combination with a polyphenol, since the allergen inactivating performance becomes synergistically high, the amount thereof added can be small, the texture of an application product is excellent, thermal discoloration resistance is excellent compared with a case in which a polyphenol compound is used on its own, and degradation of the allergen inactivating performance by heat can be suppressed. Therefore, the anti-allergen agent of the present invention is particularly preferable when a processing method involving a heating step such as a drying step in fiber processing or a step of kneading into a resin is used.

The anti-allergen agent of the present invention preferably comprises an inorganic solid acid and a polyphenol compound, and with regard to the ratio by weight of the inorganic solid acid and the polyphenol compound contained, it is preferable for there to be a defined proportion or greater of the inorganic solid acid (for there to be a defined proportion or less of the polyphenol compound) since the synergistic effect is high, the allergen inactivating performance is high, and coloration due to the polyphenol compound is suppressed. Furthermore, it is preferable for there to be a defined proportion or less of the inorganic solid acid since a high synergistic effect of the allergen inactivating performance with a polyphenol compound can be obtained. Therefore, the inorganic solid acid/polyphenol compound ratio by weight of the anti-allergen agent of the present invention is preferably 5/95 to 90/10, more preferably 20/80 to 80/20, and yet more preferably 60/40 to 80/20.

The inorganic solid acid and the polyphenol compound in the present invention exhibit synergistic effects simply by the use thereof in combination, but a state in which the polyphenol compound is present in the vicinity of the surface of the inorganic solid acid is preferable. A step of making a polyphenol compound be present in the vicinity of the surface of an inorganic solid acid is called compositing. As a method for compositing an inorganic solid acid and a polyphenol compound, there are a method in which an aqueous solution of a polyphenol is prepared and applied to an inorganic solid acid by coating, spraying, immersion, etc., a method in which compositing is carried out using compositing equipment such as a mortar, a ball mill, a ribbon mixer, etc., a method in which a precursor of a polyphenol compound is attached to the surface of an inorganic solid acid and converted into a polyphenol, etc.

With regard to the form of the anti-allergen agent in the present invention, there are powder form, clump form, tabular form, fiber form, etc., and in order to apply it to processing into various materials and configurations powder form is preferable. In the case of a powder form, the average particle size is preferably 0.01 to 50 μm, and more preferably 0.02 to 20 μm. A powder having an average particle size of at least 0.01 μm has the advantage of ease of handling since it does not easily reaggregate and, furthermore, when dispersed in a surface treatment agent such as a binder and used as a coating composition particles having an average particle size of no greater than 50 μm are preferable since they have good dispersibility, do not degrade the texture of a coated product, and do not easily cause thread breakage when kneaded with a fiber.

The color tone of the anti-allergen agent in the present invention is not limited, but in order to apply it to processing of various materials or configurations, white or a pale color having low yellowness is preferable.

With regard to a preferred yellowness, the YI value in accordance with JIS-K7103-1977 is preferably 50 or less, more preferably 20 or less, and yet more preferably 15 or less.

When a solid acid and a polyphenol compound are used in combination, compared with a conventional anti-allergen agent the anti-allergen agent in the present invention is excellent in terms of suppressing discoloration due to the polyphenol compound. For example, when tannic acid is made into an aqueous solution, the color changes over time, and there is a problem when it is used as an anti-allergen agent in a coating agent or a product that is exposed to view. However, since the above-mentioned anti-allergen agent hardly changes in color over time, it can be used in a product that is exposed to view, etc.

The anti-allergen agent of the present invention has water resistance, an anti-allergen product employing same also exhibits water resistance in terms of being washed away by rain water or water during washing, laundering, etc., and the anti-allergen effect can be exhibited sustainably.

The anti-allergen effect of the present invention is evaluated by a sandwich ELISA method, which is widely known as a method for detecting/quantifying an antigen, and is expressed as the percentage allergen inactivation shown in Equation 1. The initial amount of allergen means the amount of allergen used in an ELISA evaluation, and the amount of allergen remaining means the amount of allergen after contacting a sample. Furthermore, the allergen inactivation referred to in the present invention means suppressing a reaction between the allergen and a specific antibody, and the higher the percentage allergen inactivation, the more preferable it is. Specifically, the percentage allergen inactivation is preferably 50% or greater, more preferably 90% or greater, and yet more preferably 99% or greater.

Percentage allergen inactivation=(1−amount of allergen remaining/initial amount of allergen)×100(%) <Equation 1>

When a test target is an article comprising another substance in addition to an anti-allergen agent, such as a coating agent containing an anti-allergen agent, a resin kneaded with an anti-allergen agent, or a fiber having an anti-allergen agent attached thereto, a blank test is carried out using the same article except that the anti-allergen agent is excluded, and other measurement results may be standardized so that the percentage allergen inactivation of the blank test becomes 0. In this case, as long as it is stated that a measurement result is standardized, the value after standardization may be used as the percentage allergen inactivation.

The usage of the anti-allergen agent of the present invention is not particularly limited, and it may be made into a composition by mixing with another component or compositing with another material as appropriate according to the intended application. For example, it may be used in various configurations such as powder, powder-containing dispersion, powder-containing particles, powder-containing paint, powder-containing fiber, powder-containing paper, powder-containing plastic, powder-containing film, and powder-containing aerosol and, furthermore, if necessary various types of additives and materials such as a deodorant, an antimicrobial agent, an antifungal agent, a flame retardant, a corrosion inhibitor, a fertilizer, and a building material may be used in combination. Furthermore, addition to a material for which there is a possibility of it being in human contact, such as for example resin, paper, plastic, rubber, glass, metal, concrete, timber, paint, fiber, leather, or stone, makes it possible to inactivate allergens in a living space.

Among these application methods, those involving the use of an anti-allergen coating composition or an anti-allergen resin composition are preferable, and these two compositions are collectively called anti-allergen compositions. Among the two compositions, the coating composition is more preferable since the effect can easily be exhibited by concentrating a relatively small amount of an allergen agent on the surface of an article.

With regard to the anti-allergen coating agent, which is one of the above-mentioned anti-allergen compositions, the anti-allergen agent of the present invention is used in the form of a coating composition comprising a fixing agent, which is generally called a binder. This coating composition may comprise, in addition to a binder, another additive, and before processing the composition into an article it may be diluted with a solvent or water. From the viewpoint of ease of dispersion and good storage stability, the concentration of the anti-allergen agent contained in the composition is preferably 0.5 to 50 wt %, and more preferably 1 to 30 wt %. Since an anti-allergen effect is usually exhibited by contact between an anti-allergen agent and an allergen on the surface of an article, fixing an anti-allergen agent to the surface of an article by means of the above-mentioned coating composition is preferable because a large effect can be obtained with a smaller amount of anti-allergen agent.

The binder used in the coating composition in the present invention is not particularly limited, and examples thereof are listed as below. That is, there are a natural resin, a natural resin derivative, a phenolic resin, a xylene resin, a urea resin, a melamine resin, a ketone resin, a coumarone/indene resin, a petroleum resin, a terpene resin, cyclized rubber, chlorinated rubber, an alkyd resin, a polyamide resin, polyvinyl chloride, an acrylic resin, a vinyl chloride/vinyl acetate copolymer resin, polyvinyl acetate, polyvinyl alcohol, polyvinyl butyral, chlorinated polypropylene, a styrene resin, an epoxy resin, urethane, a cellulose derivative, etc. Among them, an acrylic resin, polyvinyl chloride, and a vinyl chloride/vinyl acetate copolymer resin are preferable, and an emulsion type resin is particularly preferable since it is less polluting and easy to handle.

Furthermore, those that can be used as an additive include a pigment such as zinc oxide or titanium oxide, a dye, an antioxidant, a light stabilizer, a flame retardant, an antistatic agent, a foaming agent, an impact modifier, glass fiber, a lubricant such as a metallic soap, a desiccant, an extending agent, a coupling agent, a nucleating agent, a flowability improving agent, a deodorant, wood flour, a fungicide, an antifoulant, a corrosion inhibitor, a metal powder, a UV absorber, and a UV shielding agent, and any thereof may be used preferably.

As a method for processing an article or fiber using a coating composition comprising the anti-allergen agent of the present invention, there is a method in which an article, a fiber product, or a material or fiber as a raw material therefor is coated with, immersed in, or sprayed with the composition as it is or a diluted liquid thereof. When a fiber is processed, there are various fibers that can be processed; examples thereof include natural fibers such as cotton, silk, and wool, synthetic fibers such as polyester, PET (polyethylene terephthalate), nylon, and acrylonitrile, semi-synthetic fibers such as triacetate and diacetate, and recycled fibers such as viscose rayon, and a composite fiber employing two or more types of the above fibers may be used. Moreover, use with a nonwoven fabric employing polyethylene, polypropylene, etc. is also possible. A method for processing fiber or a fiber product with the anti-allergen agent of the present invention is not particularly limited; there are an immersion treatment, a printing treatment, a spraying treatment, etc., and processing is completed by drying the fiber containing the composition. A drying method may employ any method such as natural drying, hot air drying, or vacuum drying, and an anti-allergen agent may be fixed to a fiber preferably by a drying method involving natural drying or heating preferably at a temperature between 50° C. and 120° C. preferably for 5 minutes to 2 hours.

The amount of the anti-allergen agent of the present invention attached to an article, a fiber product, or a raw material therefor is preferably 0.1 wt % or greater of the entire composition and more preferably 0.5 wt % or greater, and when used as a coating composition, the amount thereof is preferably at least 0.1 g per m² of the surface area thereof since an obvious effect can easily be exhibited. The amount of coating composition attached is preferably no greater than 20 g per m² of the surface area thereof from the viewpoint of economic reasons and impairment of the physical properties, texture, color, etc. of an article or fiber product to which it is added. Therefore, when used as a coating composition, the amount thereof attached is preferably 0.1 g to 20 g per m² of the surface area of an object, more preferably 0.5 g to 10 g, and yet more preferably 1 g to 5 g.

The anti-allergen resin composition, which is one of the anti-allergen compositions of the present invention, may easily be obtained by combining the anti-allergen agent of the present invention with a resin. The type of resin that can be used in the anti-allergen resin composition is not particularly limited; any of a natural resin, a synthetic resin, and a semi-synthetic resin may be used and, moreover, either of a thermoplastic resin or a thermosetting resin may be used.

Specifically, the resin may be any of a resin for molding, a resin for fiber, and a rubber-form resin, and examples thereof include resins for molding or fiber such as polyethylene, polypropylene, vinyl chloride, an ABS resin, an AS resin, an MBS resin, a nylon resin, polyester, polyvinylidene chloride, polystyrene, polyacetal, polycarbonate, PBT, an acrylic resin, a fluorine resin, a polyurethane elastomer, a polyester elastomer, a melamine resin, a urea resin, a tetrafluoroethylene resin, an unsaturated polyester resin, rayon, a cellulose acetate resin, an acrylic resin, polyvinyl alcohol, cupra, a triacetate resin, and a vinylidene resin, and rubber-form resins such as natural rubber, silicone rubber, styrene butadiene rubber, ethylene propylene rubber, fluorine rubber, nitrile rubber, chlorosulfonated polyethylene rubber, butadiene rubber, synthetic natural rubber, butyl rubber, urethane rubber, and acrylic rubber. Moreover, in addition to a resin component, various types of additives may be added. Examples of additives that can be used include a pigment such as zinc oxide or titanium oxide, a dye, an antioxidant, a light stabilizer, a flame retardant, an antistatic agent, a foaming agent, an impact modifier, glass fiber, a lubricant such as a metallic soap, a desiccant, an extending agent, a coupling agent, a nucleating agent, a flowability improving agent, a deodorant, wood flour, a fungicide, an antifoulant, a corrosion inhibitor, a metal powder, a UV absorber, and a UV shielding agent, and any thereof may be used preferably.

A method for producing a resin composition by combining the anti-allergen agent of the present invention with a resin may employ any known method. For example, there are (1) a method in which an attachment agent for making it easy for an anti-allergen agent powder to become attached to a resin or a dispersant for improving the dispersibility of an anti-allergen agent powder is used, and a pellet-form resin or a powder-form resin is directly mixed by a mixer, (2) a method in which mixing is carried out as described above, molding into pellets is carried out using an extruder, and the resulting molding is then combined with a pellet-form resin, (3) a method in which an anti-allergen agent is molded into high concentration pellet form using a wax, and the resulting pellet-form molding is then combined with a pellet-form resin, (4) a method in which a paste-form composition is prepared by dispersion-mixing an anti-allergen agent in a highly viscous liquid such as a polyol, and this paste is then combined with a pellet-form resin, etc.

Molding of the above-mentioned resin composition may employ any known molding technique and mechanical equipment commensurate with the properties of various types of resins, and preparation may be carried out easily by a method involving mixing, incorporating, or kneading while heating and applying pressure or vacuum at an appropriate temperature or pressure; specific procedures therefor may employ standard methods, and moldings can be obtained in various forms such as clump form, sponge form, film form, sheet form, thread form, pipe form, or a composite form thereof.

With regard to the usage of the anti-allergen agent of the present invention, other than the above-mentioned composition, resin composition, and resin molding, it may be used as it is, as a mixture with another component as appropriate, or as a composite with another material, according to the intended application in which it is necessary to suppress an allergen. For example, it may be used in any form such as powder form, powder dispersion form, granular form, aerosol form, or liquid form.

The anti-allergen agent of the present invention may be utilized in various fields where suppression of an allergen is required, that is, interior goods, bedding, filters, furniture, vehicle interior goods, fiber products, house building products, paper products, toys, leather products, toiletry products, and other products. Examples thereof include interior goods such as carpets, curtains, wall paper, tatami, screen paper, floor wax, and calendars, bedding such as duvets, beds, sheets, pillows, and pillow covers, filters for air cleaners or air conditioners, furniture such as sofas and chairs, vehicle interior goods such as child seats and passenger seats, dustbags for vacuum cleaners, clothing, masks, soft toys, and kitchen goods, but the examples are not limited thereto.

In accordance with the present invention, there can be provided an anti-allergen agent that has excellent heat resistance, low coloration, excellent processability, and excellent water resistance, an anti-allergen product, and a method for processing same.

### EXAMPLES

The present invention is explained in further detail by reference to Examples described below, but the Examples should not be construed as limiting the present invention.

Average particle size referred to in the Examples means a median diameter obtained by measurement using a laser diffraction particle size distribution analyzer (MALVERN MASTERSIZER model 2000). Furthermore, % denotes wt %.

Measurement of acid strength was carried out by weighing 0.1 g of a sample in a test tube, adding 2 mL of benzene and two drops of a 0.1% benzene solution of an indicator (0.1% ethanol solution for crystal violet), lightly shaking so as to mix them, and examining for change of color. Since the acid strength of a solid acid can be considered to be equal to or less than the highest acid strength for which change in color of the indicator is observed (lowest pKa value) and greater than the lowest acid strength for which change in color of the indicator is not observed (highest pKa), this range was recorded as the pKa value. The indicators used were methyl red (pKa=4.8), 4-phenylazo-1-naphthylamine (pKa=4), dimethyl yellow (pKa=3.3), 4-phenylazo-diphenylamine (pKa=1.5), crystal violet (pKa=0.8), dicinnamylacetone (pKa=−3), benzalacetophenone (pKa=−5.6), and anthraquinone (pKa=−8.2).

Water content of an anti-allergen agent was measured by allowing a sample to stand in a constant temperature and constant humidity chamber at a temperature of 25° C. and a relative humidity of 60% for 3 days. About 5 g of the sample was weighed (weighed to a precision of 0.1 mg) into an aluminum cup that had been taken to constant mass in a dryer at 250° C. for 1 hour, was dried in a dryer at 250° C. for 2 hours, and was then weighed again (weighed to a precision of 0.1 mg), the water content of the anti-allergen agent being defined by the quotient, expressed as %, obtained by dividing the decrease due to drying by the weight before drying.

Anti-allergen effects were evaluated by a sandwich ELISA method employing Dermatophagoides farinae allergen (allergen generally called DerfII) and Cryptomeria japonica pollen allergen (allergen generally called Cryj1). The test procedure when Dermatophagoides farinae allergen was used was as follows. An antibody-coated well was prepared by a standard method using a Dermatophagoides farinae allergen (DerfII)-specific antibody (15E11 antibody, Asahi Breweries, Ltd.).

1 mg or 10 mg of a sample was weighed, and 500 μL of Dermatophagoides farinae allergen (DerfII) prepared at 40 ng/mL using an antigen diluent was added thereto. The mixture was stirred well so as to contact the sample with the allergen and was then subjected to centrifugation, the supernatant was recovered, added to 15E11 antibody-coated wells that had been treated with a blocking agent, and allowed to stand at room temperature. After 1 hour, the sample was discarded, each well was washed with washing buffer, 200 ng/mL of horse radish peroxidase-labeled anti-DerfII monoclonal antibody 13A4PO (Asahi Breweries, Ltd.) that had been diluted with washing buffer was added to each well, and the wells were allowed to stand at room temperature. After 1 hour, the antibody liquid was discarded, each well was washed with washing buffer, a substrate liquid was added to each well, and the wells were allowed to stand at room temperature. After 30 minutes, 2N sulfuric acid was added so as to stop the reaction, and the absorbance at 490 nm was measured. The results were expressed as percentage allergen inactivation of various samples by determining the relationship between amount of allergen and absorbance from an evaluation carried out without using a sample, determining the amount of allergen remaining from the absorbance obtained when evaluating various types of samples, and calculating from equation 1.

Percentage allergen inactivation=(1−amount of allergen remaining/initial amount of allergen)×100(%) <Equation 1>

The test procedure for the sandwich ELISA method when Cryptomeria japonica pollen allergen was used was as follows. An antibody-coated well was prepared by a standard method using a Cryptomeria japonica pollen allergen (Cryj1)-specific antibody (Anti-Cryj1 mAb013, Seikagaku Corporation).

1 mg or 10 mg of a sample was weighed, and 500 μL of Cryptomeria japonica pollen allergen (Cryj1) prepared at 10 ng/mL using an antigen diluent was added thereto. The mixture was stirred well so as to contact the sample with the allergen and was then subjected to centrifugation, the supernatant was recovered, added to Anti-Cryj1 mAb013 antibody-coated wells that had been treated with a blocking agent, and allowed to stand at room temperature. After 1 hour, the sample was discarded, each well was washed with washing buffer, 250 ng/mL of horse radish peroxidase-labeled anti-Cryj1 monoclonal antibody 053 (Seikagaku Corporation) that had been diluted with washing buffer was added to each well, and the wells were allowed to stand at room temperature. After 2 hours, the antibody liquid was discarded, each well was washed with washing buffer, a substrate liquid was added to each well, and the wells were allowed to stand at room temperature. After 10 minutes, 2N sulfuric acid was added so as to stop the reaction, and the absorbance at 490 nm was measured. The results are expressed as percentage allergen inactivation of various types of samples by calculating from Equation 1 by the same method as for Dermatophagoides farinae allergen.

The anti-allergen effects of a processed fiber product were evaluated as percentage anti-allergen inactivation from Equation 1 above by measuring absorbance by the same ELISA method as for a solid acid powder using Dermatophagoides farinae allergen (Den) as the allergen and dividing 9 cm³ of fiber into 8, and comparing with the absorbance when a fiber product having no solid acid added thereto was used.

The anti-allergen effects of a resin film were evaluated as percentage anti-allergen inactivation from Equation 1 above by measuring absorbance by the same ELISA method as described above using Dermatophagoides farinae allergen (DerfII) as the allergen and dividing 9 cm² of film into 8, and comparing with the absorbance when a film having no anti-allergen agent added thereto was used.

### Example 1

In Example 1, the percentage anti-allergen inactivation was evaluated using 10 mg of sample.

### Example 1-1

### Lamellar Zirconium Phosphate

A lamellar zirconium phosphate was obtained by adding a 15% zirconium oxychloride aqueous solution to a 75% phosphoric acid aqueous solution, carrying out refluxing by heating for 24 hours, then filtering a precipitate, washing it with water, drying, and grinding. The lamellar zirconium phosphate thus obtained was subjected to measurement of color tone, average particle size, water content, acid strength, and mite allergen inactivating effect and Cryptomeria japonica allergen inactivating effect by the ELISA method, and the results are shown in Table 1.

### Example 1-2

### Reticulated Zirconium Phosphate

0.1 mol of oxalic acid dihydrate, 0.2 mol of zirconium oxychloride octahydrate, and 0.1 mol of ammonium chloride were dissolved in 300 mL of ion exchanged water, and 0.3 mol of phosphoric acid was then added thereto while stirring. The pH of this solution was adjusted to 2.7 using 28% aqueous ammonia, and it was then stirred at 98° C. for 14 hours. Subsequently, a precipitate thus obtained was washed well and calcined at 700° C., thus giving a reticulated zirconium phosphate. The reticulated zirconium phosphate thus obtained was subjected to measurement of color tone, average particle size, water content, acid strength, and mite allergen inactivating effect and Cryptomeria japonica allergen inactivating effect by the ELISA method, and the results are shown in Table 1.

### Example 1-3

### H-Exchanged ZSM-5 Type Zeolite

A commercial zeolite ZSM-5 (EX122, Mizusawa Industrial Chemicals, Ltd) was immersed in an aqueous solution of hydrochloric acid, then filtered, washed with water, dried, and ground, thus preparing an H-exchanged ZSM-5 type zeolite solid acid. The H-exchanged ZSM-5 type zeolite thus obtained was subjected to measurement of color tone, average particle size, water content, acid strength, and mite allergen inactivating effect and Cryptomeria japonica allergen inactivating effect by the ELISA method, and the results are shown in Table 1.

### Example 1-4

### Antimonic Acid

Antimonic acid was obtained by adding water to antimony pentachloride followed by aging at 70° C. The antimonic acid thus obtained was subjected to measurement of color tone, average particle size, water content, acid strength, and mite allergen inactivating effect and Cryptomeria japonica allergen inactivating effect by the ELISA method, and the results are shown in Table 1.

### Example 1-5

### Silica-Alumina

A precipitate obtained using water glass and aluminum nitrate as starting materials was calcined at 500° C. and then ground, thus preparing silica-alumina. The silica-alumina thus obtained was subjected to measurement of color tone, average particle size, water content, acid strength, and mite allergen inactivating effect and Cryptomeria japonica allergen inactivating effect by the ELISA method, and the results are shown in Table 1.

### Example 1-6

### H-Exchanged Y Type Zeolite

A commercial zeolite Y (Mizuka Sieves Y400, Mizusawa Industrial Chemicals, Ltd.) was immersed in an aqueous solution of hydrochloric acid, then filtered, washed with water, dried, and ground, thus preparing an H-exchanged Y type zeolite solid acid.

The H-exchanged Y type zeolite thus obtained was subjected to measurement of color tone, average particle size, water content, acid strength, and mite allergen inactivating effect by the ELISA method, and the results are shown in Table 1.

### Comparative Example 1-1

### Composite Mineral Comprising Silicon Dioxide, Zinc Oxide, and Aluminum Oxide

A commercial composite mineral comprising silicon dioxide, zinc oxide, and aluminum oxide (Mizukanite HP, Mizusawa Industrial Chemicals, Ltd.) was subjected to measurement of color tone, average particle size, acid strength, and mite allergen inactivating effect by the ELISA method, and the results are shown in Table 1.

### Comparative Example 1-2

### A Type Zeolite

A commercial zeolite A (Siliton B, Mizusawa Industrial Chemicals, Ltd.) was subjected to measurement of color tone, average particle size, acid strength, and mite allergen inactivating effect and Cryptomeria japonica allergen inactivating effect by the ELISA method, and the results are shown in Table 1.

### Comparative Example 1-3

### X Type Zeolite

A commercial zeolite X (CPT-30, Mizusawa Industrial Chemicals, Ltd.) was subjected to measurement of color tone, average particle size, acid strength, and mite allergen inactivating effect and Cryptomeria japonica allergen inactivating effect by the ELISA method, and the results are shown in Table 1.

### Comparative Example 1-4

### ZSM-5 Type Zeolite

A commercial zeolite ZSM-5 (EX122, Mizusawa Industrial Chemicals, Ltd.) was subjected to measurement of color tone, average particle size, acid strength, and mite allergen inactivating effect by the ELISA method, and the results are shown in Table 1.

### Comparative Example 1-5

### Hydrotalcite

A commercial hydrotalcite (HT-P, Sakai Chemical Industry Co., Ltd.) was subjected to measurement of color tone, average particle size, acid strength, and mite allergen inactivating effect by the ELISA method, and the results are shown in Table 1.

### Comparative Example 1-6

### Aluminum Oxide

A reagent-grade aluminum oxide was subjected to measurement of color tone, average particle size, acid strength, and mite allergen inactivating effect by the ELISA method, and the results are shown in Table 1.

### Comparative Example 1-7

### Zinc Oxide

A commercial zinc oxide (zinc oxide type 2, Sakai Chemical Industry Co., Ltd.) was subjected to measurement of color tone, average particle size, acid strength, and mite allergen inactivating effect by the ELISA method, and the results are shown in Table 1.

TABLE 1

| | | | | | | Percentage | |
|---|---|---|---|---|---|---|---|
| | | | Average | Water | | allergen | |
| | | Color | particle | content | | inactivation (%) | |
| | Component | tone | size (μm) | (%) | pKa | DerfII | Cryj1 |
| Example 1-1 | Lamellar | White | 1.0 | 4.6 | −8.2 to −5.6 | Greater | Greater |
| | zirconium | | | | | than | than |
| | phosphate | | | | | 99.9 | 99.9 |
| Example 1-2 | Reticulated | White | 0.9 | 2.4 | −5.6 to −3.0 | Greater | Greater |
| | zirconium | | | | | than | than |
| | phosphate | | | | | 99.9 | 99.9 |
| Example 1-3 | H-exchanged | White | 3.0 | 10.7 | −5.6 to −3.0 | 97.1 | 91.3 |
| | ZSM-5 type | | | | | | |
| | zeolite | | | | | | |
| Example 1-4 | Antimonic acid | White | 1.8 | 20.7 | 0.8 to 1.5 | Greater | 87 |
| | | | | | | than | |
| | | | | | | 99.9 | |
| Example 1-5 | Silica-alumina | White | 5.4 | 9.1 | 0.8 to 1.5 | 98.7 | 97.2 |
| Example 1-6 | H-exchanged Y | White | 2.1 | 17.0 | 3.3 to 4.0 | 72.5 | N.D. |
| | type zeolite | | | | | | |
| Comparative | Composite | White | 4.0 | N.D. | 4.0 to 4.8 | 45 | N.D. |
| Example 1-1 | mineral | | | | | | |
| | comprising | | | | | | |
| | silicon dioxide, | | | | | | |
| | zinc oxide, and | | | | | | |
| | aluminum oxide | | | | | | |
| Comparative | A type zeolite | White | 3.5 | N.D. | Greater | 34.5 | N.D. |
| Example 1-2 | | | | | than | | |
| | | | | | 4.8 | | |
| Comparative | X type zeolite | White | 3.1 | N.D. | Greater | 33.8 | N.D. |
| Example 1-3 | | | | | than | | |
| | | | | | 4.8 | | |
| Comparative | ZSM-5 type | White | 2.1 | N.D. | Greater | 11.9 | N.D. |
| Example 1-4 | zeolite | | | | than | | |
| | | | | | 4.8 | | |
| Comparative | Hydrotalcite | White | 4.4 | N.D. | Greater | 12.8 | N.D. |
| Example 1-5 | | | | | than | | |
| | | | | | 4.8 | | |
| Comparative | Aluminum | White | 1.2 | N.D. | Greater | 2.5 | 0 |
| Example 1-6 | oxide | | | | than | | |
| | | | | | 4.8 | | |
| Comparative | Zinc oxide | White | 0.4 | N.D. | Greater | 11.7 | 0 |
| Example 1-7 | | | | | than | | |
| | | | | | 4.8 | | |

*N.D.: not determined

From the results in Table 1, all of the solid acids of the present invention showed a percentage mite allergen inactivation of 50% or greater. In particular, lamellar zirconium phosphate, reticulated zirconium phosphate, and antimonic acid exhibited the effect of the percentage allergen inactivation being greater than 99.9%, and were truly excellent as anti-allergen agents.

Furthermore, in the case of Cryptomeria japonica pollen allergen, as in the case of mites, the solid acids of the present invention exhibited a high percentage allergen inactivation and were truly excellent as anti-allergen agents. On the other hand, in the Comparative Examples where the pKa was larger than 4.0, hardly any anti-allergen activity was exhibited.

### Example 1-8

### Evaluation of Anti-Allergen Activity of Fiber-Fixed Solid Acid

The H-exchanged ZSM-5 type zeolite solid acid of Example 1-3 and an acrylic emulsion binder (Kesmon Binder KB1300, solids content 45%, Toagosei Co., Ltd.) were mixed at a solids content ratio by weight of 10:3, and applied to a fabric (components: cotton/acrylic fiber=1/1) by immersion and drying, thus preparing an anti-allergen fabric having an amount fixed of 10 g/m². The allergen inactivating effect of the anti-allergen fabric was measured, and the result is shown in Table 2.

### Example 1-9

### Evaluation of Anti-Allergen Activity of Fiber-Fixed Solid Acid

The H-exchanged ZSM-5 type zeolite solid acid of Example 1-3 and Kesmon Binder KB1300 (solids content 45%, Toagosei Co., Ltd.) were mixed at a solids content ratio by weight of 10:3, applied to a fabric (components: cotton/acrylic fiber=1/1) by immersion for 5 minutes, and then dried at 120° C. for 30 minutes, thus preparing an anti-allergen fabric having an amount fixed of 15 g/m³. The allergen inactivating effect of the anti-allergen fabric was measured, and the result is shown in Table 2.

### Comparative Example 1-8

### Evaluation of Anti-Allergen Activity of Fiber

A comparative fabric was prepared by the same processing method as in Example 1-8 without using an H-exchanged ZSM-5 type zeolite solid acid. The allergen inactivating effect of the comparative fabric was measured, and the result is shown in Table 2.

TABLE 2

| | Solid acid and | Percentage allergen |
|---|---|---|
| | amount processed | inactivation (%) |
| Example 1-8 | H-exchanged ZSM-5 type zeolite | 99.3 |
| | 10 g/m² | |
| Example 1-9 | H-exchanged ZSM-5 type zeolite | 100 |
| | 15 g/m² | |
| Comparative | Fabric alone, no binder | 12 |
| Example | | |
| 1-8 | | |

From the results in Table 2, the anti-allergen processed fabrics to which a solid acid was attached exhibited a percentage allergen inactivation of 99% or greater. Therefore, the performance of the anti-allergen products formed by post-processing a fiber with a solid acid was excellent.

### Example 1-10

### Evaluation of Heat Resistance of Fiber-Fixed Solid Acid

An anti-allergen fabric was prepared by the same method as in Example 1-9 and heated at 200° C. for 2 hours, the allergen inactivating effect and color change of the anti-allergen fabric were measured, and the results are shown in Table 3.

TABLE 3

| | | Percentage allergen | |
|---|---|---|---|
| | Solid acid and | inactivation | |
| | amount processed | (%) | Color change |
| Example | H-exchanged ZSM-5 | 99% | No color |
| 1-10 | type zeolite | | change |
| | 15 g/m² | | |

From the results of Table 3, since the anti-allergen processed fabric to which a solid acid was attached exhibited a sufficiently high percentage allergen inactivation even when heat was applied and in addition did not exhibit a change in color, the anti-allergen product formed by post-processing a fiber with the solid acid had excellent heat resistance.

### Example 2

Evaluation of percentage anti-allergen inactivation in Example 2 was carried out using 1 mg of a sample unless otherwise specified.

### Example 2-1

### Anti-Allergen Agent (1)

A lamellar zirconium phosphate was obtained by adding a 15% zirconium oxychloride aqueous solution to a 75% phosphoric acid aqueous solution, carrying out refluxing by heating for 24 hours, then filtering a precipitate, washing it with water, drying, and grinding. The lamellar zirconium phosphate thus obtained and tannic acid were mixed at a mixing ratio by weight of 7/3, composited by means of a ball mill for 3 hours, and ground by means of a rotor speed mill, thus giving anti-allergen agent (1). The anti-allergen agent thus obtained was subjected to measurement of average particle size, yellowness, water content, and mite allergen inactivating effect and Cryptomeria japonica allergen inactivating effect by the ELISA method, and the results are shown in Table 4.

### Example 2-2

### Anti-Allergen Agent (2)

A lamellar zirconium phosphate prepared in the same manner as in Example 2-1 and tannic acid were mixed at a mixing ratio by weight of 6/4, composited by means of a ball mill for 3 hours, and ground by means of a rotor speed mill, thus giving anti-allergen agent (2). The anti-allergen agent thus obtained was subjected to measurement of yellowness, water content, and mite allergen inactivating effect and Cryptomeria japonica allergen inactivating effect by the ELISA method, and the results are shown in Table 4.

### Example 2-3

### Anti-Allergen Agent (3)

A precipitate obtained using water glass and aluminum nitrate as starting materials was calcined at 500° C. and then ground, thus preparing silica-alumina. The silica-alumina thus obtained and tannic acid were mixed at a mixing ratio by weight of 8/2, composited by means of a ball mill for 3 hours, and ground by means of a rotor speed mill, thus giving anti-allergen agent (3). The anti-allergen agent thus obtained was subjected to measurement of yellowness, water content, and mite allergen inactivating effect by the ELISA method, and the results are shown in Table 4.

### Example 2-4

### Anti-Allergen Agent (4)

Silica-alumina prepared in the same manner as in Example 2-3 and tannic acid were mixed at a mixing ratio by weight of 7/3, composited by means of a ball mill for 3 hours, and ground by means of a rotor speed mill, thus giving anti-allergen agent (4). The anti-allergen agent thus obtained was subjected to measurement of average particle size, yellowness, water content, and mite allergen inactivating effect by the ELISA method, and the results are shown in Table 4.

### Example 2-5

### Anti-Allergen Agent (5)

Silica-alumina prepared in the same manner as in Example 2-3 and tannic acid were mixed at a mixing ratio by weight of 6/4, composited by means of a ball mill for 3 hours, and ground by means of a rotor speed mill, thus giving anti-allergen agent (5). The anti-allergen agent thus obtained was subjected to measurement of yellowness, water content, and mite allergen inactivating effect by the ELISA method, and the results are shown in Table 4.

### Example 2-6

### Anti-Allergen Agent (6)

A lamellar zirconium phosphate prepared in the same manner as in Example 2-1 and tannic acid were mixed at a mixing ratio by weight of 3/97, composited by means of a ball mill for 3 hours, and ground by means of a rotor speed mill, thus giving anti-allergen agent (6). The anti-allergen agent thus obtained was subjected to measurement of yellowness and mite allergen inactivating effect by the ELISA method, and the results are shown in Table 4.

### Example 2-7

### Anti-Allergen Agent (7)

A lamellar zirconium phosphate was obtained by adding a 15% zirconium oxychloride aqueous solution to a 75% phosphoric acid aqueous solution, carrying out refluxing by heating for 24 hours, then filtering a precipitate, washing it with water, drying, and grinding. The lamellar zirconium phosphate thus obtained was subjected to measurement of yellowness, average particle size, water content, acid strength, and mite allergen inactivating effect and Cryptomeria japonica allergen inactivating effect by the ELISA method, and results are shown in Table 4. The amount of anti-allergen agent was 10 mg.

### Example 2-8

### Anti-Allergen Agent (8)

A precipitate obtained using water glass and aluminum nitrate as starting materials was calcined at 500° C. and then ground, thus preparing silica-alumina. The silica-alumina thus obtained was subjected to measurement of yellowness, average particle size, water content, acid strength, and mite allergen inactivating effect and Cryptomeria japonica allergen inactivating effect by the ELISA method, and the results are shown in Table 4. The amount of anti-allergen agent was 10 mg.

### Comparative Example 2-1

### Tannic Acid

Tannic acid was subjected to measurement of average particle size, yellowness, and mite allergen inactivating effect and Cryptomeria japonica allergen inactivating effect by the ELISA method, and the results are shown in Table 4.

TABLE 4

| | Component | | Average | | | Percentage | |
|---|---|---|---|---|---|---|---|
| | (amount of anti- | | particle | Water | | allergen | |
| | allergen agent 1 mg | | size | content | | inactivation (%) | |
| | in principle) | Yellowness | (μm) | (%) | pka | DerfII | Cryj1 |
| Example 2-1 | Lamellar | 11.6 | 5.1 | 7.3 | — | 99.1 | Greater |
| | zirconium | | | | | | than |
| | phosphate/tannic | | | | | | 99.9 |
| | acid = | | | | | | |
| | 7/3 | | | | | | |
| Example 2-2 | Lamellar | 15.5 | — | 7.2 | — | 99.6 | Greater |
| | zirconium | | | | | | than |
| | phosphate/tannic | | | | | | 99.9 |
| | acid = | | | | | | |
| | 6/4 | | | | | | |
| Example 2-3 | Silica- | 9.2 | — | 18.9 | — | 98.7 | — |
| | alumina/tannic | | | | | | |
| | acid = | | | | | | |
| | 8/2 | | | | | | |
| Example 2-4 | Silica- | 11.9 | 12.5 | 17.3 | — | 96.9 | — |
| | alumina/tannic | | | | | | |
| | acid = | | | | | | |
| | 7/3 | | | | | | |
| Example 2-5 | Silica- | 15.7 | — | 15 | — | 99 | — |
| | alumina/tannic | | | | | | |
| | acid = | | | | | | |
| | 6/4 | | | | | | |
| Example 2-6 | Lamellar | 30.9 | — | — | — | 95.7 | — |
| | zirconium | | | | | | |
| | phosphate/tannic | | | | | | |
| | acid = | | | | | | |
| | 3/97 | | | | | | |
| Example 2-7 | Lamellar | 0.76 | 1 | 4.6 | −8.2 to −5.6 | Greater | Greater |
| | zirconium | | | | | than | than |
| | phosphate | | | | | 99.9 | 99.9 |
| | (10 mg) | | | | | | |
| Example 2-8 | Silica-alumina | 0.84 | 5.4 | 9.1 | 0.8 to 1.5 | 98.7 | 97.2 |
| | (10 mg) | | | | | | |
| Comparative | Tannic acid | 71.28 | 110 | — | — | 95 | 97 |
| Example 2-1 | | | | | | | |

In Table 4, the notation ‘-’ means that measurement was not carried out.

In Table 4, when comparing the allergen inactivating performance of an anti-allergen agent comprising an inorganic solid acid and tannic acid with that of the inorganic solid acid on its own or tannic acid on its own, the anti-allergen agent comprising an inorganic solid acid and tannic acid exhibited a higher percentage allergen inactivation than that of tannic acid on its own. Furthermore, the percentage allergen inactivation exhibited by the use of 1 mg of the anti-allergen agent of Example 2-1, which was a composite, was the same level as that exhibited by the use of 10 mg of the inorganic solid acid on its own in Example 2-7, the amount of Example 2-1 being 1/10 of that of Example 2-7, and showed a higher level than that of Comparative Example 2-1, this suggesting that the composite of the present invention exhibits a synergistic effect with respect to the anti-allergen effect.

In particular, anti-allergen agents (1) and (2) (Examples 2-1 and 2-2) comprising lamellar zirconium phosphate and tannic acid exhibited a high percentage allergen inactivation of 99% or greater for mite and Cryptomeria japonica allergens. Furthermore, compared with Examples 2-7 and 2-8, they exhibited a high percentage anti-allergen inactivation with one tenth of the amount, and the anti-allergen performance of anti-allergen agents (1) and (2) was truly excellent. Moreover, the yellowness of tannic acid on its own was 71.8, which is extremely high, but the yellowness of the anti-allergen agent of the present invention was low, which is preferable in terms of coloration for an anti-allergen product.

### Example 2-9

### Evaluation of Anti-Allergen Activity of Fiber-Fixed Anti-Allergen Agent

The anti-allergen agent of Example 2-1 and an acrylic emulsion binder (Kesmon Binder KB1300, solids content 45%, Toagosei Co., Ltd.) were mixed at a solids content ratio by weight of 10/3, and applied to a fabric (components: cotton/acrylic fiber=1/1) by immersion and then drying at 120° C. for 15 minutes, thus preparing an anti-allergen fabric having an amount fixed of 4.3 g/m². The anti-allergen fabric was subjected to measurement of allergen inactivating effect. In this test, since the allergen decreases over the course of the test due to adsorption, etc. on the fabric even when an anti-allergen agent is not used, the measurement result was standardized so that the percentage allergen inactivation of the blank test of Comparative Example 2-4 employing no anti-allergen agent was 0, and is shown in Table 5.

### Example 2-10

### Evaluation of Water Resistance of Fiber-Fixed Anti-Allergen Agent

An anti-allergen fabric having the anti-allergen agent of Example 2-1 fixed thereto was prepared by the same method as in Example 2-9, placed in a 1 L plastic container charged with 500 mL of ion exchanged water so that it was immersed, washed by agitating for 1 minute, and then air-dried. Subsequently, the result of measurement of the allergen inactivating effect of the anti-allergen fabric was standardized by the same method as in Example 2-9, and is shown in Table 5.

### Example 2-11

### Evaluation of Anti-Allergen Activity of Fiber-Fixed Anti-Allergen Agent

The procedure of Example 2-9 was repeated except that the anti-allergen agent of Example 2-1 was changed to the anti-allergen agent of Example 2-6 and the amount fixed was changed to 4 g/m². The result of measurement of the allergen inactivating effect of the anti-allergen fabric was standardized by the same method as in Example 2-9, and is shown in Table 5.

### Example 2-12

An anti-allergen fabric having the anti-allergen agent of Example 2-6 fixed thereto was prepared by the same method as in Example 2-11, placed in a 1 L plastic container charged with 500 mL of ion exchanged water so that it was immersed, washed by agitating for 1 minute, and then air-dried. Subsequently, the result of measurement of the allergen inactivating effect of the anti-allergen fabric was standardized by the same method as in Example 2-9, and is shown in Table 5.

### Example 2-13

### Evaluation of Anti-Allergen Activity of Fiber-Fixed Solid Acid

The lamellar zirconium phosphate solid acid of Example 2-7 and an acrylic emulsion binder (Kesmon Binder KB1300, solids content 45%, Toagosei Co., Ltd.) were mixed at a solids content ratio by weight of 10/3, applied to a fabric (components: cotton/acrylic fiber=1/1) by immersion, and then dried at 120° C. for 15 minutes, thus preparing an anti-allergen fabric having an amount fixed of 4.6 g/m². Subsequently, the result of measurement of the allergen inactivating effect was standardized by the same method as in Example 2-9, and is shown in Table 5.

### Example 2-14

### Evaluation of Water Resistance of Fiber-Fixed Solid Acid

An anti-allergen fabric having the solid acid of Example 2-7 fixed thereto was prepared by the same method as in Example 2-13, placed in a 1 L plastic container charged with 500 mL of ion exchanged water so that it was immersed, washed by agitating for 1 minute, and then air-dried. Subsequently, the result of measurement of the allergen inactivating effect of the anti-allergen fabric was standardized by the same method as in Example 2-13, and is shown in Table 5.

### Comparative Example 2-2

### Evaluation of Anti-Allergen Activity of Fiber-Fixed Tannic Acid

Tannic acid and an acrylic emulsion binder (Kesmon Binder KB1300, solids content 45%, Toagosei Co., Ltd.) were mixed at a solids content ratio by weight of 10/3, and applied to a fabric (components: cotton/acrylic fiber=1/1) by immersion and then dried at 120° C. for 15 minutes, thus preparing a comparative fabric having an amount fixed of 4.6 g/m². The result of measurement of the allergen inactivating effect of the comparative fabric was standardized, and is shown in Table 5.

### Comparative Example 2-3

### Evaluation of Water Resistance of Fiber-Fixed Tannic Acid

A comparative fabric having tannic acid fixed thereto was prepared by the same method as in Comparative Example 2-2, placed in a 1 L plastic container charged with 500 mL of ion exchanged water so that it was immersed, washed by agitating for 1 minute, and then air-dried. Subsequently, the result of measurement of the allergen inactivating effect of the comparative fabric was standardized by the same method as in Comparative Example 2-2, and is shown in Table 5.

### Comparative Example 2-4

### Evaluation of Anti-Allergen Activity of Blank Test

A comparative fabric was prepared by the same processing method as in Example 2-9 without using an anti-allergen agent. The allergen inactivating effect of the comparative fabric was measured, and the percentage inactivation measurement results of Examples 2-9 to 2-14 and Comparative Examples 2-2 to 2-5 were standardized so that the percentage inactivation of the above comparative fabric had a figure of 0, and are shown in Table 5. The percentage allergen inactivation of Comparative Example 2-4 is therefore 0.

### Comparative Example 2-5

### Evaluation of Water Resistance of Fiber on its Own

A comparative fabric was prepared by the same processing method as in Example 2-9 without using the anti-allergen agent of the present invention, and subjected to washing. The result of measurement of the allergen inactivating effect of the comparative fabric was standardized, and is shown in Table 5.

TABLE 5

| | Solid acid and | Percentage allergen |
|---|---|---|
| | amount processed | Inactivation (%) |
| Example 2-9 | Lamellar zirconium | Greater than 99.9 |
| | phosphate/tannic acid = | |
| | 7/3 4.3 g/m² | |
| Example 2-10 | After washing | Greater than 99.9 |
| Example 2-11 | Lamellar zirconium | Greater than 99.9 |
| | phosphate/tannic acid = | |
| | 3/97 4 g/m² | |
| Example 2-12 | After washing | Less than 10 |
| Example 2-13 | Lamellar zirconium | 94.2 |
| | phosphate | |
| | 4.6 mg/m | |
| Example 2-14 | After washing | 95.1 |
| Comparative | Tannic acid | Greater than 99.9 |
| Example 2-2 | 4.6 mg/m² | |
| Comparative | Tannic acid | 0 |
| Example 2-3 | (after washing) | |
| Comparative | Fabric binder | 0 |
| Example 2-4 | (standardized to 0) | |
| Comparative | Fabric binder | 0 |
| Example 2-5 | (after washing) | |

In Table 5, the percentage allergen inactivation of the fabric of Comparative Example 2-4 on its own was defined as 0%, and the results of the Examples and other Comparative Examples in Table 5 were standardized based thereon.

The anti-allergen processed fabric to which anti-allergen agent (1) of Example 2-9 of the present invention had been attached had an allergen inactivation of greater than 99.9%. Furthermore, in the case of tannic acid, the activity disappeared after washing, but in the case of the processed fabric to which anti-allergen agent (1) of the present invention had been attached, the anti-allergen effect did not degrade, thus exhibiting water resistance, and the percentage allergen inactivation of Example 2-10, which was tested after washing, was greater than 99.9%. Therefore, the anti-allergen product formed by post-processing a fiber with the anti-allergen agent of the present invention has excellent allergen inactivating performance and excellent water resistance.

### Example 2-15

### Evaluation of Heat Resistance of Fiber-Fixed Solid Acid

An anti-allergen fabric was prepared by the same method as in Example 2-9 and subjected to heating at 200° C. for 2 hours, and the allergen inactivating effect and color change of the anti-allergen fabric were then measured, the results thereof being shown in Table 6.

### Comparative Example 2-6

An anti-allergen fabric was prepared by the same method as in Comparative Example 2-2 and subjected to heating at 200° C. for 2 hours, and the allergen inactivating effect and color change of the anti-allergen fabric were then measured, the results thereof being shown in Table 6.

TABLE 6

| | | Percentage | |
|---|---|---|---|
| | Solid acid and | allergen | |
| | amount processed | inactivation (%) | Color change |
| Example 2-15 | Lamellar | Greater than | Changed to |
| | zirconium | 99.9 | very pale brown |
| | phosphate/tannic | | |
| | acid = | | |
| | 7/3 4.3 g/m² | | |
| Comparative | Tannic acid | 96% | Dark brown |
| Example 2-6 | 4.6 mg/m² | | |

From the results of Table 6, since the anti-allergen processed fabric to which the anti-allergen agent of the present invention was attached exhibited a sufficiently high percentage allergen inactivation even when heat was applied and in addition exhibited hardly any change in color, the anti-allergen product formed by post-processing a fiber with the anti-allergen agent of the present invention has excellent heat resistance. On the other hand, one processed with tannic acid showed a severe color change, and was not practical.

### Example 2-16

### Discoloration Test and Anti-Allergen Activity

1 mg of anti-allergen agent (1) of Example 2-1 was placed in 0.5 mL of PBS (pH 7.29, containing 0.1% Tween 20 and 0.001% BSA) and allowed to stand at room temperature for 3 days, color change and allergen inactivating effect were then measured, and the results are shown in Table 7.

### Example 2-17

### Discoloration Test

1 mg of anti-allergen agent (1) of Example 2-1 was placed in 0.5 mL of ion exchanged water (pH 6.37) and allowed to stand at room temperature for 7 days, color change was then examined, and the result is shown in Table 7.

### Comparative Example 2-7

### Discoloration Test and Anti-Allergen Activity

1 mg of tannic acid was placed in 0.5 mL of PBS (pH 7.29, containing 0.1% Tween 20 and 0.001% BSA) and allowed to stand at room temperature for 3 days, color change and allergen inactivating effect were then measured, and the results are shown in Table 7.

### Comparative Example 2-8

### Discoloration Test

1 mg of tannic acid was placed in 0.5 mL of ion exchanged water (pH 6.37) and allowed to stand at room temperature for 7 days, color change was examined, and the result is shown in Table 7.

TABLE 7

| | Anti-allergen agent | | | Percentage |
|---|---|---|---|---|
| | used (1 mg)/ | Color of solution | Color of solution | allergen |
| | evaluated liquid | (before evaluation) | (after evaluation) | inactivation (%) |
| Example 2-16 | Example 2-1/PBS | Colorless | Colorless | 98.8 |
| | | | (no color change) | |
| Example 2-17 | Example 2-1/water | Colorless | Colorless | — |
| | | | (no color change) | |
| Comparative | Tannic acid/PBS | Colorless | Brown | Less than 50 |
| Example 2-7 | | | (color changed) | |
| Comparative | Tannic acid/water | Colorless | Brown | — |
| Example 2-8 | | | (color changed) | |

In Table 7, the notation ‘-’ means that measurement was not carried out.

From the results of Table 7, tannic acid underwent color change in an aqueous solution state and degradation in allergen inactivating performance was observed, but the anti-allergen agent of the present invention of Examples 2-16 and 2-17 did not show a color change in an aqueous solution state and exhibited a high percentage allergen inactivation. Therefore, the anti-allergen agent of the present invention has excellent durability since color change does not occur even in aqueous solution and there is little effect on allergen inactivating performance.

### Evaluation of Anti-Allergen Activity of Anti-Allergen Agent Kneaded with Resin

### Example 2-18

In Example 2-18, the above-mentioned lamellar zirconium phosphate solid acid was mixed with a polyethylene resin powder (HI-ZEX 1300JPU, Prime Polymer Co., Ltd.) at 30% of the total weight, heated at 180° C. for 5 minutes, air-cooled for 4 minutes, then sandwiched between polytetrafluoroethylene sheets and flattened out with a pressure of 150 kg/cm² using a desktop press, thus preparing a 0.2 to 0.3 mm thick film. The film thus prepared was white.

### Example 2-19

In Example 2-19, anti-allergen agent (1), that is, a composite of lamellar zirconium phosphate/tannic acid=7/3 was mixed as in Example 2-18 at 30% of the total resin composition, thus preparing a film. The film thus prepared was white as in Example 2-18, but when compared with the sheet of Example 2-18 side by side, it was tinged with yellow.

### Example 2-20

In Example 2-20, anti-allergen agent (1), that is, a composite of lamellar zirconium phosphate/tannic acid=7/3 was mixed as in Example 2-18 at 10% of the total resin composition, thus preparing a film. The film thus prepared was white as in Example 2-18, but when compared with the sheet of Example 2-18 side by side, it was slightly tinged with yellow, but it was closer to white than Example 2-19.

### Comparative Example 2-9

In Comparative Example 2-9, a film was prepared in the same manner as in Example 2-18 except that tannic acid was mixed at 30% of the total resin composition. The film thus prepared was dark brown.

### Comparative Example 2-10

In Comparative Example 2-10, a film was prepared in the same manner as in Example 2-18 except that tannic acid was mixed at 10% of the total resin composition. The film thus prepared was dark brown.

### Comparative Example 2-11

In Comparative Example 2-11, a film was prepared using polyethylene resin alone.

Evaluation of the allergen inactivating performance of the films was carried out by the ELISA method using Dermatophagoides farinae allergen (DerfII) as described above. In Comparative Example 2-9, in which tannic acid was 30%, as soon as the film was contacted with an allergen liquid, brown tannic acid leached out into the allergen liquid, and the entire liquid changed color to brown. Since this cannot be said to be an evaluation of the film, the result of Comparative Example 2-9 was not recorded. Since the result of Comparative Example 2-11 is given as 0, standardization was not carried out, and the evaluation results are shown as it is in Table 8.

TABLE 8

| | | Percentage | |
|---|---|---|---|
| | | allergen | |
| | Solid acid and | inactivation | Appearance of |
| | amount processed | (%) | film |
| Example 2-18 | Lamellar | Greater | White |
| | zirconium | than 99.9 | |
| | phosphate 30%/PE | | |
| Example 2-19 | Lamellar | Greater | White (touch |
| | zirconium | than 99.9 | of yellow) |
| | phosphate/tannic | | |
| | acid = | | |
| | 7/3 30%/PE | | |
| Example 2-20 | Lamellar | 67.4 | White (slight |
| | zirconium | | touch of yellow) |
| | phosphate/tannic | | |
| | acid = | | |
| | 7/3 10%/PE | | |
| Comparative | Tannic acid | Not evaluated | Dark brown |
| Example 2-9 | 30%/PE | | |
| Comparative | Tannic acid | Less | Dark brown |
| Example 2-10 | 10%/PE | than 50 | |
| Comparative | PE resin on its own | 0 | Colorless |
| Example 2-11 | | | transparent |

The results of Table 8 suggest that when tannic acid, which is a polyphenol, was used on its own, not only was an intensive color change caused by heating during melting of a resin, but also the allergen inactivating performance was lost. On the other hand, a resin film formed by mixing lamellar zirconium phosphate, which is the anti-allergen agent of the present invention, is resistant to color change by heating, and has excellent durability such that there is little effect on the allergen inactivating performance. Furthermore, although there is a possibility of the anti-allergen agent formed by compositing the inorganic solid acid of the present invention with tannic acid causing a slight color change by heating, compared with the Comparative Examples where tannic acid was used on its own, resistance to discoloration was excellent, and the heat resistance in terms of allergen inactivating performance was also excellent.

### INDUSTRIAL APPLICABILITY

In accordance with use of the anti-allergen agent of the present invention, it becomes possible to impart a function of inactivating allergens such as pollen or mites to a material related to a human living space such as a fiber product or filter, and an anti-allergen product can be produced simply at low cost.

1. An anti-allergen agent comprising as an active ingredient an inorganic solid acid.

2. The anti-allergen agent according to claim 1, wherein the inorganic solid acid has an acid strength as pKa of 4.0 or less.

3. The anti-allergen agent according to claim 1, wherein the inorganic solid acid is at least one selected from the group consisting of zirconium phosphate, aluminum phosphate, tin phosphate, cerium phosphate, titanium phosphate, an H-exchanged Y type zeolite, an H-exchanged ZSM-5 type zeolite, antimonic acid, an SiO₂—Al₂O₃ composite oxide, an SiO₂—TiO₂ composite oxide, an SiO₂—ZrO composite oxide, an SiO₂—Ga₂O₃ composite oxide, a TiO₂—Al₂O₃ composite oxide, a TiO₂—ZrO composite oxide, a TiO₂—SnO composite oxide, a TiO₂—ZnO composite oxide, and magnesium silicate.

4. The anti-allergen agent according to claim 1, wherein the anti-allergen agent further comprises a polyphenol compound.

5. The anti-allergen agent according to claim 4, wherein the inorganic solid acid is contained at 5 to 90 wt % relative to the total amount of the inorganic solid acid and the polyphenol compound.

6. The anti-allergen agent according to claim 4, wherein the polyphenol compound is tannic acid.

7. An anti-allergen composition comprising the anti-allergen agent according to claim 1.

8. A method for processing an anti-allergen product, employing the anti-allergen composition according to claim 7.

9. An anti-allergen product that has been processed by the method for processing an anti-allergen product according to claim 8.

10. The anti-allergen agent according to claim 2, wherein the inorganic solid acid has an acid strength as pKa of 1.5 or less.

11. The anti-allergen agent according to claim 1, wherein the inorganic solid acid is at least one selected from the group consisting of zirconium phosphate, an H-exchanged ZSM-5 type zeolite, an H-exchanged Y type zeolite, and an SiO₂—Al₂O₃ composite oxide.

12. The anti-allergen agent according to claim 1, wherein the inorganic solid acid is zirconium phosphate.
